(19) United States
(12) Patent Application Publication
(10) Pub. No.: US 2015/0246213 A1
(43) Pub. Date: Sep. 3, 2015

(54) Apparatus For Applying Topical Substance

(71) Applicant: Meridian Industries, Inc., Milwaukee, WI (US)

(72) Inventors: Douglas J. Arnold, Franklin, WI (US); Jeffrey G. Maxon, Waldo, WI (US); Mark A. Oman, Belgium, WI (US); Steven W. Rabus, Belgium, WI (US); Frank C. DeGuire, Brookfield, WI (US)

(73) Assignee: Meridian Industries, Inc., Milwaukee, WI (US)

(21) Appl. No.: 14/193,276

(22) Filed: Feb. 28, 2014

(51) Int. Cl. A61F 13/40; A61F 13/38

(52) U.S. Cl. CPC A61M 35/006; A61F 13/38; A61M 2210/04

(57) ABSTRACT

An applicator apparatus for supporting a pad to be applied to a body is described. The apparatus includes a handle, an apparatus head attached to an end of the handle, the apparatus head including a front face and a rear face, the front face being configured to receive a disposable pad for application to the body, the disposable pad extending over substantially all of the front face and the rear face including at least one mounting hook configured to receive a portion of the disposable pad extending from the portion proximate to the front face to the rear face. The apparatus further includes the disposable pad configured for use with the apparatus head having at least one mounting hook receiving opening for receiving the at least one mounting hook on the rear face of the apparatus head.

### BACKGROUND

The present application relates to a handle and pad support plate for supporting a pad having a substance thereupon for topical application. More particularly, the present application relates to an apparatus having a handle and pad support plate, the pad support plate having a front face opposite a handle attachment point and a rear face including pad attachment means.

Users often use topical substances applied directly to their skin for a variety of reasons including treating a skin condition, moisturizing, cleaning, etc. The present application is directed to an apparatus for use in applying such a topical substance. The apparatus is configured to allow a user to apply the topical substance to their body without directly contacting the topical substance with their hands and further to allow the user to apply the topical substance to areas of their body that may be difficult to reach with their hands, such as their back. These substances are frequently in the form of lotions, creams, solutions or ointments.

A similar handle and pad support exists in the marketplace but uses Velcro on a face of the pad support that can degrade over time leave harsh edges and get clogged with pad fibers reducing the effectiveness of the support functionality. Further, integrating a Velcro pad onto an entire front face of a pad support can increase manufacturing costs and selling price.

What is needed is an apparatus for supporting a pad having a topical substance thereupon and further being configured to facilitate application of the topical substance. What is further needed is such an apparatus including pad attachment means on a rear face of a pad supporting plate.

### SUMMARY OF THE INVENTION

The present invention provides an applicator apparatus for supporting a pad to be applied to a body. The apparatus includes a handle, an apparatus head attached to an end of the handle, the apparatus head including a front face and a rear face, the front face being configured to receive a disposable pad for application to the body, the disposable pad extending over substantially all of the front face and the rear face including at least one mounting hook configured to receive a portion of the disposable pad extending from the portion proximate to the front face to the rear face. The apparatus further includes the disposable pad configured for use with the apparatus head having at least one mounting hook receiving openings for receiving the at least one mounting hook on the rear face of the apparatus head.

The present application further includes an applicator apparatus for supporting a pad to be applied to a body. The apparatus includes a handle and an apparatus head attached to an end of the handle, the apparatus head including a front face and a rear face, the front face being configured to receive a disposable pad for application to the body, the disposable pad extending over substantially all of the front face and the rear face including one mounting hook positioned on a first side of the rear face and configured to receive a portion of the disposable pad extending from the portion proximate to the front face to the rear face and at least one attachment point positioned on a second side of the rear face opposite the first side. The apparatus further includes the disposable pad configured for use with the apparatus head having at least one mounting hook receiving opening for receiving the at least one mounting hook on the rear face of the apparatus head.

The present application yet further includes an applicator apparatus for supporting a pad to be applied to a body. The apparatus includes a handle and an apparatus head attached to an end of the handle, the apparatus head including a front face, at least two side walls, and a rear face. The front face is configured to receive a disposable pad for application to the body, the disposable pad extending over substantially all of the front face, the side walls extend from the front face in the direction of the rear face and including at least one mounting hook configured to receive a portion of the disposable pad extending from the portion proximate to the front face to the rear face, and the rear face including an attachment point for attaching the handle to the apparatus head. The apparatus further includes the disposable pad configured for use with the apparatus head having at least one mounting hook receiving openings for receiving the at least one mounting hook on the rear face of the apparatus head.

The present application yet further includes an applicator apparatus for supporting a pad to be applied to a body. The apparatus includes a handle and an apparatus head attached to an end of the handle, the apparatus head including a front face and a rear face, the front thee being configured to receive a disposable pad for application to the body, and the rear face including at least one of a mounting hook configured to receive a portion of the disposable pad extending from the portion proximate to the front face to the rear face and an attachment point. The apparatus further includes the disposable pad including a topical substance configured for use with the apparatus head and sized to cover substantially all of the front face and be attached to the apparatus head using an attachment on the roar face of the apparatus head.

These particular features and advantages may apply to only some embodiments falling within the claims and thus do not define the scope of the invention.

### BRIEF DESCRIPTION OF THE FIGURES

FIG. 1 is an isometric view of an applicator apparatus for supporting a pad to be applied to a body, according to an exemplary embodiment;

FIG. 2A is a top down view of the apparatus head of the apparatus of FIG. 1, according to an exemplary embodiment;

FIGS. 2B-2E are isometric views of the apparatus of FIG. 1 and a disposable pad, according to an exemplary embodiment;

FIGS. 3A-3E are depictions of the geometric shapes of the apparatus head of the apparatus of FIG. 1, according to an exemplary embodiment;

FIGS. 4A-4D are views of an alternative embodiment of the apparatus of FIG. 1 depicting a front face of apparatus head having a textured surface, according to an exemplary embodiment;

FIG. 5A-5E are side isometric views of another alternative embodiment of the apparatus of FIG. 1 depicting varying configuration of the apparatus head, mounting hooks and attachment points; and

FIGS. 6A-6C are views of an alternative embodiment of the apparatus of FIG. 1 depicting a front face of apparatus head having a soft surface, according to an exemplary embodiment.

### DETAILED DESCRIPTION OF AN EXEMPLARY EMBODIMENT

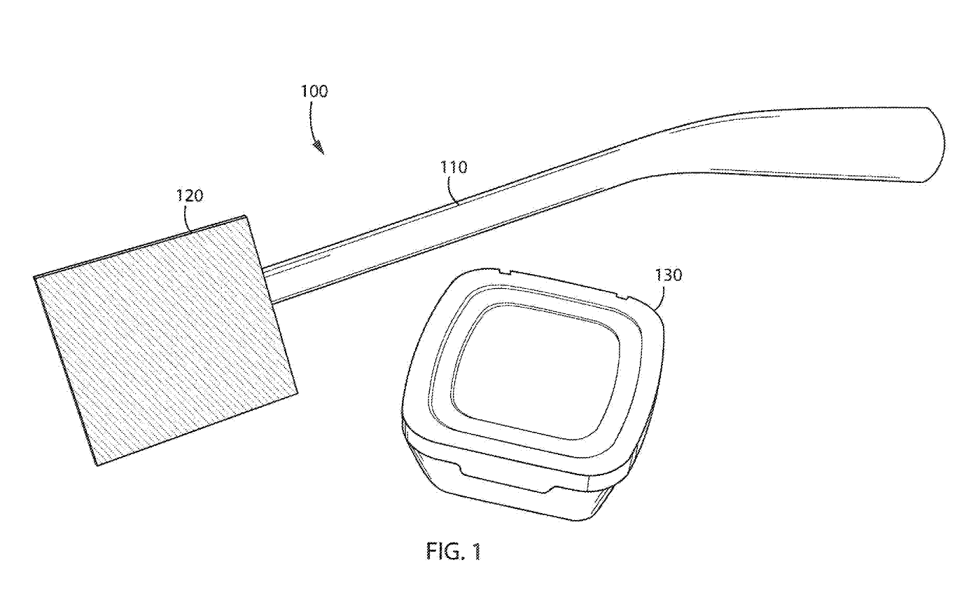

Referring first to FIG. 1, a topical substance application kit 100 including an applicator handle 110, an applicator head 120, and a pad container 130 are shown, according to an exemplary embodiment. Kit 100 may be provided as a single kit including all of the components and/or as separate components to be used in combination for applying a topical substance. For example, additional pad containers 130 may be available separately as a user uses a supply of pads contained within the pad container 130.

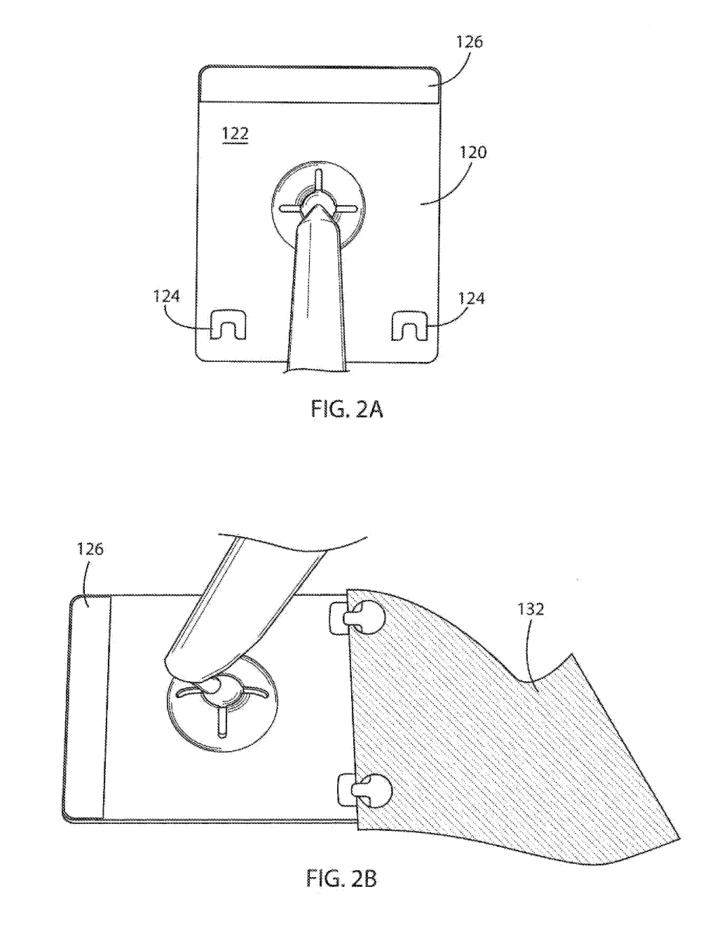

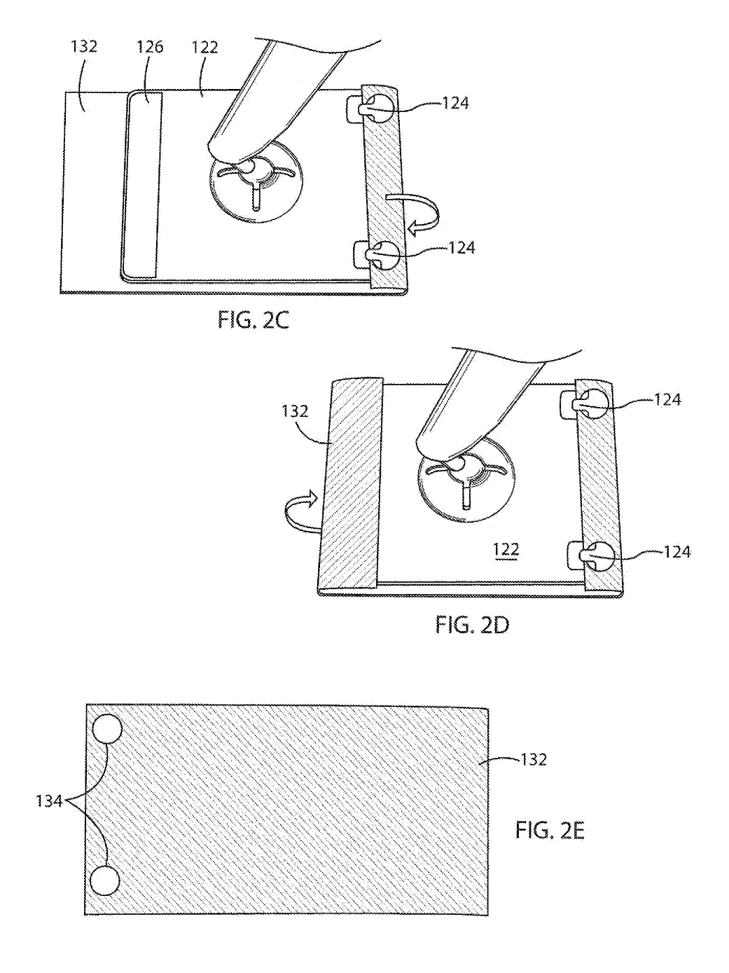

Applicator handle 100 may be provided in any one of a variety of configurations. For example, applicator handle 100 may be provided as a single piece wand or a multi-piece wand. Further, applicator handle 100 may be provided as a rigid wand or be configured to include a telescoping feature allowing a user to extend or shorten the length of the handle 100. Yet further, applicator handle 100 may be provided in different configurations depending on the intended usage. For example, applicator handle 100 may be substantially straight or may be provided with one or more bolded portions along some or all of the length of the handle 100 to facilitate its intended use. Applicator head 100 is configured to include an applicator head attachment point attaching applicator head 120 to applicator handle 110, shown and described below with reference to FIG. 2. Applicator head 120 is configured to support a topical substance application pad allowing a user holding the applicator handle 110 and is described in further detail below with reference to FIGS. 2-6.

Pad container 130 may be any type of jar or other container including a stack of pads where the user is intended to remove a single pad for use. Examples include facial tissues, make up removal pads, skin cleansing pads, topical treatment pad, etc. The pads enclosed in this type of container may be pads that have been specially treated with a topical substance to perform a specific function. The treatment typically includes a liquid solution that is absorbed and retained with each article of the stack of articles. Examples including disposable cleaning pads having a cleaning topical substance, medicated pads having a medicine topical substance, etc. Alternatively, the pads may be dry pads and/or dry pads configured to receive the topical substance when it is applied by a user prior to use.

Pad container 130 may be provided in a variety of shapes dependent on the shape of the pads to be used on combination with applicator head 120 as described below. For example, pad container 100 may be rectangular as shown in FIG. 1, may be square, may be roughly triangular, etc.

According to exemplary embodiment, container 130 may be configured to have a top opening sized to receive the applicator head 120. The top opening of container 130 may further be configured to allow a user to insert applicator head 120 and/or one or more fingers to affix a top pad from the stack of pads to the applicator head 120 using attachment means as described hereinbelow.

Referring now to FIGS. 2A-2E, a rear thee 122 of applicator head 120 including attachment hooks 124 and secondary attachment points 126 are shown, according to one exemplary embodiment. Referring first to FIG. 2A, an attachment end of the handle 110 include a ball and socket type attachment to the applicator head 120. A ball and socket attachment allows the user to position the applicator head 110 against their skin independent of the angle of the handle 110. This attachment type allows the user to reach all areas of their body and still provide good contact with the pad 132, shown in FIG. 2E.

Although FIG. 2A shows a specific type of attachment between the handle 110 and the applicator head 120, it should be understood that a variety of different attachment types may be used in the alternative. For example, in the alternative, applicator 120 may be attached to handle 110 using a hinged attachment point. Further the attachment point may be directly molded into the rear face 122 of the applicator head 120.

Attachment hooks 124 may be any type of protuberance extending upward from rear face 122. Examples can include hooks, pegs, posts, and/or any other type of protuberance configured to receive and retain a first end of pad 132 as described herein. In operation, as shown in FIG. 2B, applicator head 120 is placed on a pad 132 such that the rear face 122 is facing away from the pad 132. As shown in FIG. 2C, pad holes 134 are drawn over the rear face 122 to extend over the attachment hooks 124. Accordingly, and as shown in FIG. 2D, the pad 132 is first affixed to the attachment hooks 124 at the first end of the pad 132, then drawn around the applicator from the rear face 122 and over a front face (riot shown) and back to the rear face 122. Then, the pad 132, at a second end, is attached to the rear face 122 using secondary attachment points 126. Secondary attachment points 126 may be Velcro tabs, a Velcro strip, additional protuberances, etc to receive and hold the second end of the pad 132.

Although embodiment shown in the FIGs depict use of both mounting hooks 124 and attachment points 126, it should be understood that alternative embodiments may utilize one of mounting hooks 124 or attachment points 126 on the rear face 122. Additionally, applicator head 120 may be removable from handle 110 to allow for replacement of the applicator head 120 as the head and/or attachments wear during usage.

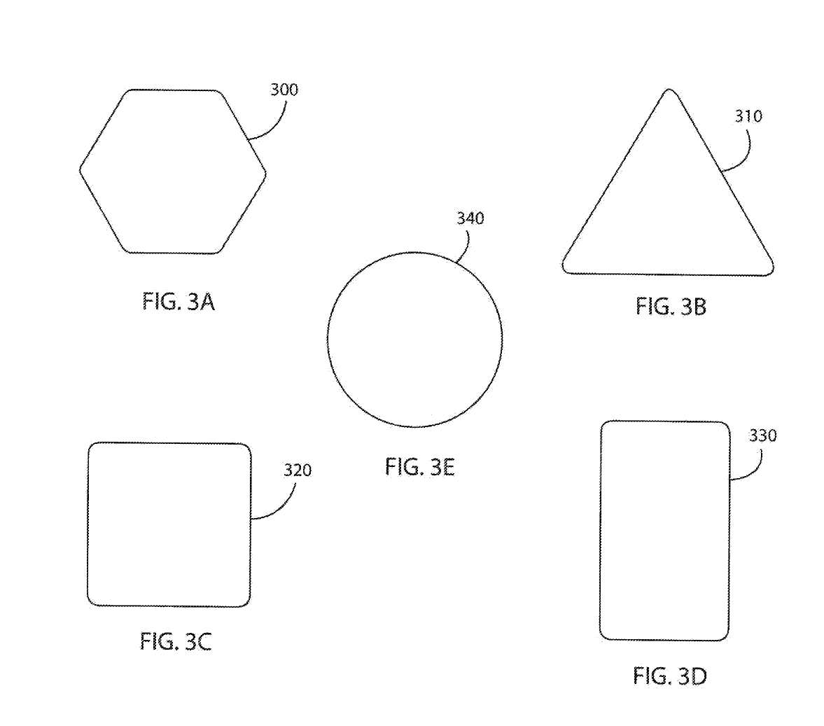

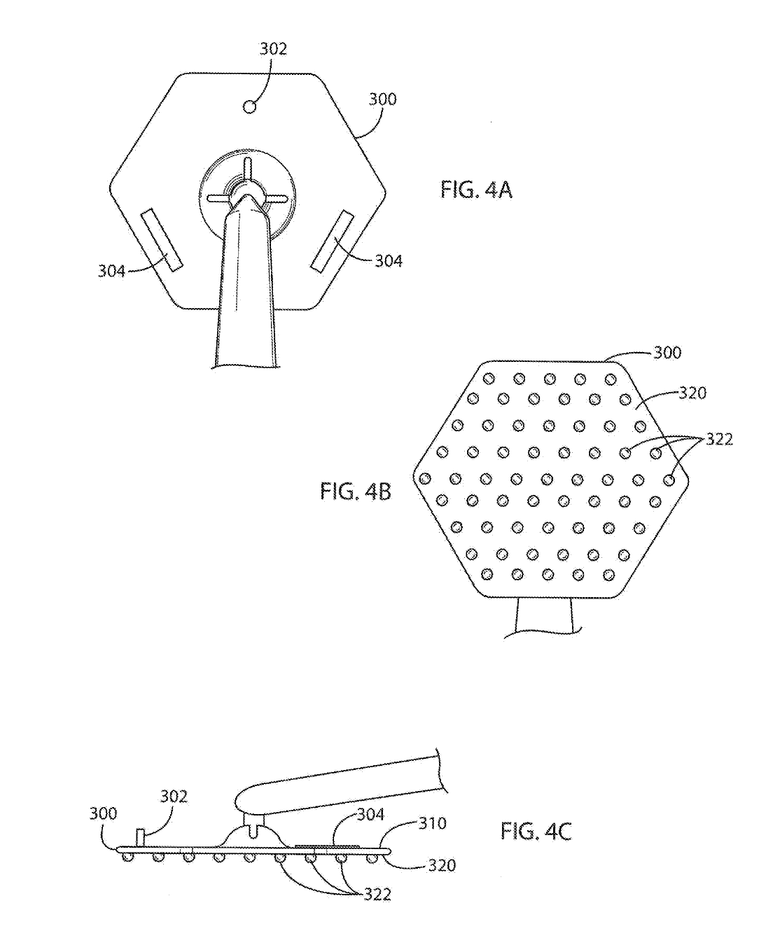

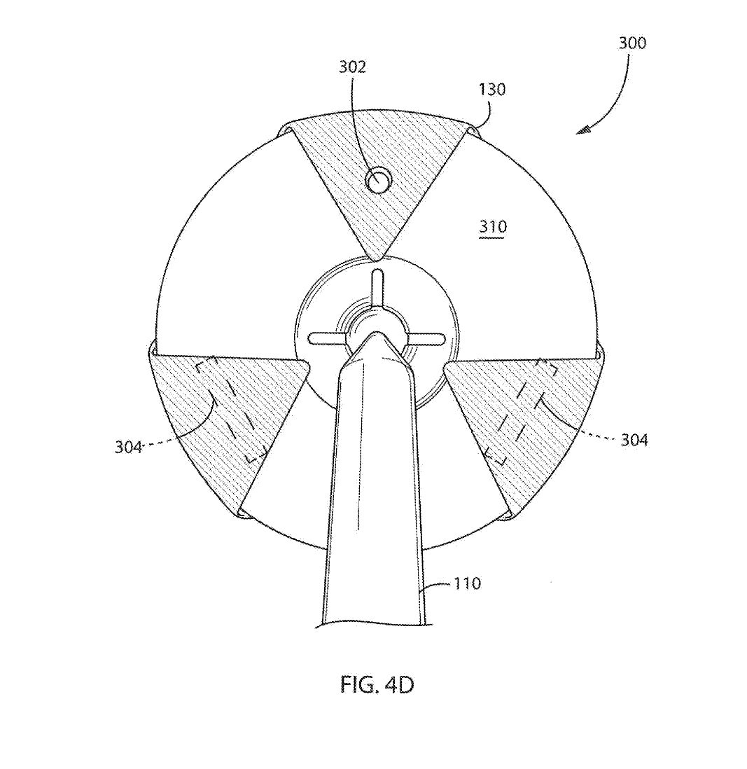

Referring now to FIGS. 3A-3D, applicator head 120 may be provided in a variety of different geometric configurations. For example, a hexagonal head 300 may be provided for use with triangular pads 132. Referring now also to FIG. 4A, using a hexagonal attachment head 300, the head may include a single mounting hook 302 configured to be placed through the mounting hole 134 of one point of a triangular shaped pad 132. Thereafter, the pad may be wrapped around the head 300 and attached to the head 300 at the second and third points of the triangular pad 132 using secondary attachment points 134, shown in the figure as Velcro strips. Additional head shapes may include a triangular head 310, as shown in FIG. 3B, a square head 320, as shown in FIG. 3C, a rectangular head 330, as shown in FIG. 3D, and a round head 340, as shown in FIG. 3E. Although specific head shapes are shown, any shape head, such as a round head, may be used to provided the advantages described herein.

Referring now to FIGS. 4A-4B, applicator head 300 includes a rear face 310 and a front face 320. According to an exemplary embodiment, means for affixing the pad 130 to the applicator head 300 are provided on the rear face 310 to allow the front face 320 to be substantially consistent. A consistent front face 320 prevents the user from feeling attachment points when the applicator head 300 is being held against their skin during use.

Front face 320 may include a smooth or textured surface. As shown in FIGS. 4B and 4C, front face 320 may include a number of smaller protuberances 322 that reduce slippage of the pad 130 when the pad 130 is being held against the skin and moved around by the user. It should be understood that any of a variety of different types of texturing may be used to provide this advantage.

Referring now to FIG. 4D, applicator head 300 may be implemented using a rounded shape but still including a rear face 310. According to an exemplary embodiment, means for affixing the pad 130 to the applicator head 300, such as mounting hook 302 and attachment point 304, are provided on the rear face 310 to allow the front face 320 to be substantially consistent. A consistent front face 320 prevents the user from feeling attachment points when the applicator head 300 is being held against their skin during use.

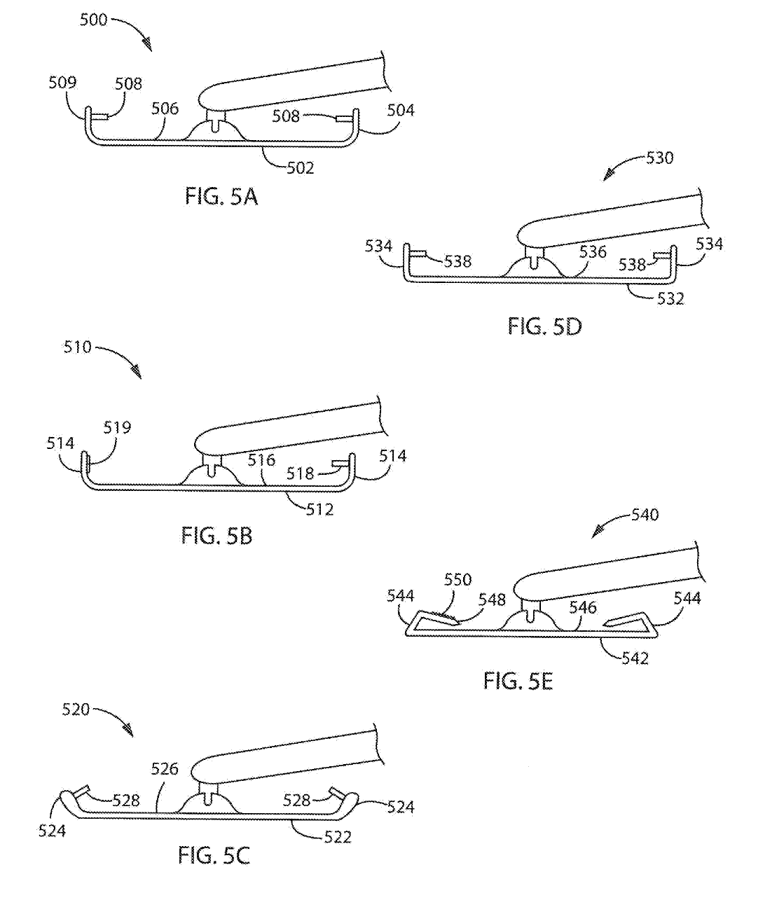

Referring now to FIGS. 5A-5E, applicator head 120 may be provided in a variety of different configurations. The applicator head may have different configurations based on intended usage, such as usage on specific body parts, and/or may have different configurations dependent on the type of pad 130 to be used, such as wet versus dry pads.

Referring now to FIG. 5A, an applicator head 500 may be configured to have a front face 502 having sidewalls 504 that extend away from the plane of the front face in the direction of the rear face 506. According to an exemplary embodiment, the transition from front face 502 to sidewalls 504 may be defined by a curve having sufficient radius to avoid sharp corners. In this embodiment, mounting hooks 508 may be provided on both inside side of sidewalls 504 proximate to the rear face 506.

Referring first to FIG. 5B, an applicator head 510 may be configured to again have a front face 512 having sidewalls 514 that extend away from the plane of the front face in the direction of the rear face 516. According to an exemplary embodiment, the transition from front face 512 to sidewalls 514 may be defined by a curve having sufficient radius to avoid sharp corners. In this embodiment, a mounting hook 518 may be provided on a first inside side of sidewalls 514 proximate to the rear face 516 and an attachment point 519 may be provided on the second inside side of sidewalls 514.

Referring now to FIG. 5C, an applicator head 520 similar to applicator head 500 may be configured to have a front face 522 having sidewalls 524 that extend away from the plane of the front face in the direction of the rear face 526. According to an exemplary embodiment, the transition from front face 522 to sidewalls 524 may be defined by a curve having sufficient radius to avoid sharp corners. Further, this embodiment, sidewalls 524 may be configured to vary in thickness from a relatively narrow thickness proximate to the juncture between sidewalls 524 and front face 522 to a relatively larger thickness away from this juncture. In this embodiment, mounting hooks 528 may be provided on both inside side of sidewalls 524 proximate to the rear face 526. The embodiment may alternatively be configured to include the mounting hook and attachment point configuration shown in FIG. 5B.

Referring now to FIG. 5D, an applicator head 530 similar to applicator head 500 may be configured to have a front face 532 having sidewalls 534 that extend away from the plane of the front face in the direction of the rear face 536. According to an exemplary embodiment, the transition from front face 532 to sidewalls 534 may be defined by a straight wall having substantially no radial transition between front face 532 and sidewalls 534. In this embodiment, mounting hooks 538 may be provided on both inside side of sidewalls 534 proximate to the rear face 536. The embodiment may alternatively be configured to include the mounting hook and attachment point configuration shown in FIG. 5B.

Referring now to FIG. 5E, an applicator head 540 similar to applicator head 500 may be configured to have a front face 542 having sidewalls 544 that extend away from the plane of the front face in the direction of the rear face 546. According to an exemplary embodiment, the transition from front face 542 to sidewalls 544 may be defined by a straight wall having substantially no radial transition between front face 542 and sidewalls 544. In this embodiment, inward extensions 548 may further be provided on the end of sidewall 544 opposite the juncture between sidewalls 544 and front face 542. Inward extensions 548 include a serrated edge end 550 on the end of each inward extension 544 opposite the juncture between inward extension 544 and sidewalls 544.

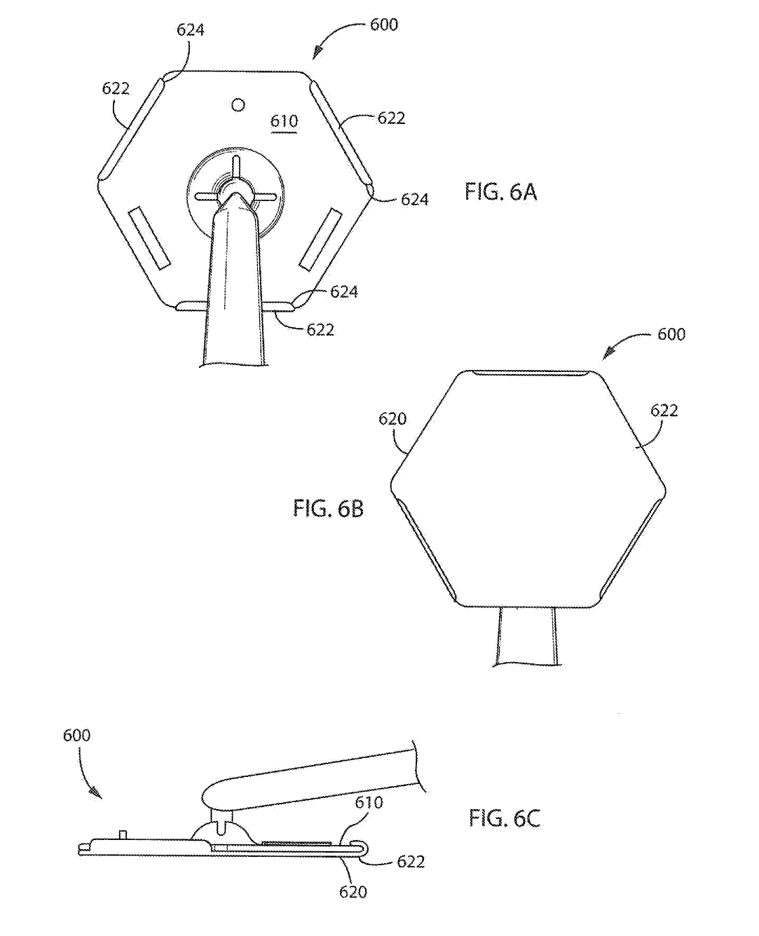

Referring now to FIGS. 6A-6C, applicator head 600 includes a rear face 610 and a front face 620. According to an exemplary embodiment, means for affixing the pad 130 to the applicator head 600 are provided on the rear face 610 to allow the front face 620 to be substantially consistent. A consistent front face 620 prevents the user from feeling attachment points when the applicator head 600 is being held against their skin during use.

Front face 620 may include a soft surface pad 622 provided by any soft material, such as a felt pad or rubbed pad. As shown in FIGS. 4B and 4C, front face 620 may include an integrally molded soft surface pad 622 over the front face 620 that reduce slippage of the pad 130 when the pad 130 is being held against the skin and moved around by the user. Molded soft surface pad 622 may be configured to wrap around the edges of front face 620 and have attachment points 624 molded into the rear face 610. Advantageously, when a pad is affixed to the applicator head 600, all of the edges of front face 620 will be covered by either wrapped pad 130 or wrapped soft surface pad 622. It should be understood that any of a variety of different types of padded material may be used to provide this advantage.

There has been disclosed herein an apparatus for applying a substance to difficult to reach portions of the body. The elements and the features of the invention are described herein with respect to obtaining those objectives; however, the basic structure of the invention may be modified to carry out those objectives. Thus, references herein to specific details of the illustrated embodiment are by way of example and not by way of limitation. It will be apparent to those skilled in the art that many modifications of the basic illustrated embodiment may be made without departing from the spirit and scope of the invention as recited by the claims.

The present invention has been described in terms of one exemplary embodiment, and it is recognized that equivalents, alternatives, and modifications, aside from those expressly stated, are possible and within the scope of the appended claims.

We claim:

1. An applicator apparatus for supporting a pad to be applied to a body, the apparatus comprising
a handle;
an apparatus head attached to an end of the handle, the apparatus head including a front face and a rear face,
the front face being configured to receive a disposable pad for application to the body, the disposable pad extending over substantially all of the front face and
the rear face including at least one mounting hook configured to receive a portion of the disposable pad extending from the portion proximate to the front face to the rear face; and
the disposable pad configured for use with the apparatus head having at least one mounting hook receiving openings for receiving the at least one mounting hook on the rear face of the apparatus head.

2. The device of claim 1, front face includes a plurality of protuberances configured to reduce movement of the disposable pad when the apparatus head is being pressed against a body part and moved.

3. The device of claim 1, wherein the rear face includes at least two mounting hooks.

4. The device of claim 3, wherein the at least two mounting hooks are spaced apart from each other sufficiently to cause tension across the disposable pad and secure the disposable pad to the mounting hooks.

5. The device of claim 1, wherein the rear face includes at least one attachment point configured to secure the disposable pad to the apparatus head after the disposable pad has been affixed to the at least one mounting hook and positioned across the front face.

6. The device of claim 1, wherein the apparatus head has a rounded configuration and the disposable pad is substantially triangular or rectangular.

7. The device of claim 1, wherein the apparatus head includes at least two sidewalls attached to the front face and extending away from the front face toward the rear face.

8. The device of claim 7, wherein the at least one mounting hook is positioned on the side wall extending toward an attachment point between the apparatus handle and the apparatus head.

9. An applicator apparatus for supporting a pad to be applied to a body, the apparatus comprising
a handle;
an apparatus head attached to an end of the handle, the apparatus head including a front face and a rear face,
the front face being configured to receive a disposable pad for application to the body, the disposable pad extending over substantially all of the front thee and
the rear face including one mounting hook positioned on a first side of the rear face and configured to receive a portion of the disposable pad extending from the portion proximate to the front face to the rear face and at least one attachment point positioned on a second side of the rear face opposite the first side; and
the disposable pad configured for use with the apparatus head having at least one mounting hook receiving opening for receiving the at least one mounting hook on the rear face of the apparatus head.

10. The device of claim 9, front face includes a plurality of protuberances configured to reduce movement of the disposable pad when the apparatus head is being pressed against a body part and moved.

11. The device of claim 9, wherein attachment point is configured to secure the disposable pad to the apparatus head after the disposable pad has been affixed to the mounting hook and positioned across the front face.

12. The device of claim 9, wherein the apparatus head includes at least two sidewalls attached to the front face and extending away from the front face toward the rear face.

13. The device of claim 12, wherein the at least one mounting hook is positioned on the side wall extending toward an attachment point between the apparatus handle and the apparatus head.

14. An applicator apparatus for supporting a pad to be applied to a body, the apparatus comprising
a handle;
an apparatus head attached to an end of the handle, the apparatus head including a front face, at least two side walls, and a rear face,
the front face being configured to receive a disposable pad for application to the body, the disposable pad extending over substantially all of the front face,
the side walls extending from the front face in the direction of the rear face and including at least one mounting hook configured to receive a portion of the disposable pad extending from the portion proximate to the front face to the rear face, and
the rear face including an attachment point for attaching the handle to the apparatus head; and
the disposable pad configured for use with the apparatus head having at least one mounting hook receiving openings for receiving the at least one mounting hook on the rear face of the apparatus head.

15. The device of claim 14, wherein the front face includes a plurality of protuberances configured to reduce movement of the disposable pad when the apparatus head is being pressed against a body part and moved.

16. The device of claim 14, wherein one side wall includes at least one attachment point configured to secure the disposable pad to the apparatus head after the disposable pad has been affixed to the at least one mounting hook and positioned across the front face.

17. The device of claim 14, wherein the apparatus head has a rounded configuration and the disposable pad is substantially triangular or rectangular.

18. The device of claim 14, wherein the at least one mounting hook is positioned on the side wall extending toward the attachment point between the apparatus handle and the apparatus head.

19. An applicator apparatus for supporting a pad to be applied to a body, the apparatus comprising
a handle;
an apparatus head attached to an end of the handle, the apparatus head including a front face and a rear face,
the front face being configured to receive a disposable pad for application to the body, and
the rear face including at least one of a mounting hook configured to receive a portion of the disposable pad extending from the portion proximate to the front face to the rear face and an attachment point; and
the disposable pad including a topical substance configured for use with the apparatus head and sized to cover substantially all of the front face and be attached to the apparatus head using an attachment on the rear face of the apparatus head.
